(19) United States
(12) Patent Application Publication
(10) Pub. No.: US 2002/0193758 A1
(43) Pub. Date: Dec. 19, 2002

(54) Product

(75) Inventor: Stefan Sandberg, Goteborg (SE)

Correspondence Address: YOUNG & THOMPSON, 745 SOUTH 23RD STREET 2ND FLOOR, ARLINGTON, VA, 22202

(73) Assignee: SCA HYGIENE PRODUCTS AB, GOTEBORG (SE)

(21) Appl. No.: 10/173,111

(22) Filed: Jun. 18, 2002

Related U.S. Application Data
(60) Provisional application No. 60/298,404, filed on Jun. 18, 2001.

(30) Foreign Application Priority Data
Jun. 18, 2001 (SE) 0102152-6

(51) Int. Cl. A61F 13/00; A61F 13/15; A61F 13/20

(52) U.S. Cl. 604/304; 604/367; 604/385.17; 604/387

(57) ABSTRACT

The present invention provides an absorbent article, selected from the group consisting of a sanitary napkin, a panty liner, and an interlabial menstruation protection device, wherein the article is suitable for administrating a pharmaceutical substance. In one embodiment, the pharmaceutical substance is suitable for treating, mitigating and/or preventing the premenstrual syndrome. The article comprises an absorbent body and has a generally elongated shape. The article has a first surface which is intended to face the wearer, and it has a second surface which is intended to face away from the wearer. The first surface, which is intended to face the wearer, is at least partially covered with a transdermal therapeutic system containing a compound suitable for treating and/or preventing the pre-menstrual syndrome, whereby said compound is transdermally administrated to the wearer when said article is worn.

The present invention relates to an absorbent product, such as a sanitary napkin or a panty liner. The absorbent product of the invention comprises a transdermal therapeutic system for administrating a pharmaceutical composition for treating the premenstrual syndrome, or PMS. The invention also relates to using said absorbent product for preparing a pharmaceutical product for treating the premenstrual syndrome.

### TECHNICAL BACKGROUND

Premenstrual syndrome (PMS) is a recurrent disorder that occurs in the luteal phase of the menstrual cycle. It is characterised by intense physical, psychologic and behavioral changes that interrupt interpersonal relationships and disrupt the lives of affected women. Up to 40% of women of childbearing age have some form of PMS, and up to 10% have severe signs and symptoms [Ugarizza et al., Nurse Pract 1998 September; 23(9): 40, 45, 49-52]. Although no single causative factor has been identified, PMS is related to ovulatory cycles and resolves at menopause. Treatment is based on the symptoms. Oral contraceptive agents, diuretics and antidepressants have been used to relieve symptoms [Daugherty, Am. Fam. Phys. 58:183-92, 197-8 (1998)].

Somatic symptoms of premenstrual syndrome include bloating, weight gain, mastalgia, abdominal discomfort and pain, lack of energy, headache, and exacerbations of chronic illnesses such as asthma allergies, epilepsy, or migraine. Commonly reported affective changes are dysphoria, irritability, anxiety, tension, aggresion, feelings of being unable to cope, and a sense of loss of control [Smith et al., Clin. Rev. Gynaecol. 1994:939-47].

As already mentioned, several therapies have been proposed in order to treat and prevent PMS. U.S. Pat. No. 5,817,819 relates to gonadotropin-releasing hormone antagonists, which, among all can be used to treat and/or prevent PMS. More specifically the invention provides optionally substituted condensed-bicyclic compounds consisting of a homo or hetero 5 to 7 membered ring and a home or hetero 5 to 7 membered ring. Compositions containing these antagonists can be administred as tablets, injectable solutions, and granules.

EP 211,502 describes a method for treating the pre-menstrual syndrome in which one or more of linoleic acid and the metabolites of linoleic acid, together with one or more of alpha-linolenic acid and the metabolites of alpha-linolenic acid are administred to a patient in need thereof. One way of administrating the above compounds is to include them in creams and lotions for topical administation through the skin.

More sophisticated transdermal systems have also been used in order to administrate pharmaceutical compounds for treating and/or preventing PMS. U.S. Pat. No. 5,904,931 discloses a transdermal therapeutic system containing sex steroids and dimethyl isosorbide. The active ingredients are contained in a polymeric matrix covered by an adhesive layer. In a preferred embodiment, the therapeutic system is a plaster. In addition to PMS the plaster can be used to treat, for example, endometriosis, gestagen-dependent tumours, benign breast diseases, menopausal symptoms, prevention of osteoporosis and regulation of the menstrual cycle.

U.S. Pat. No. 5,788,984 describes a pharmaceutical composition for transdermal administration comprising gestodene and a pharmaceutically acceptable carrier for transdermal administration and optionally an estrogenic compound. The composition can be an ointment, lotion or a spray. The composition is used as a contraceptive.

U.S. Pat. No. 5,633,009 discloses a device for transdermally administrating therapeutically effective amounts of azaspirone compounds. The azaspirone compounds are used for treating psychogenetic symptomalogy, including premenstrual syndrome. The device is a triple laminate comprising backing layer/matrix layer containing azaspirone/release (peel strip) layer.

U.S. Pat. No. 5,399,355 discloses a device similar to the one in U.S. Pat. No. 5,633,009 for transdermally administrating ergoline derivatives. Ergoline derivatives are dopaminergic agonists that can be used in order to treat, among all, premenstrual syndrome.

The transdermal devices of the above cited documents are all plasters of about 10 cm². Accordingly, such plasters are quite large, and sometimes, it is difficult for the wearer to cover the plaster. Therefore, wearing such plasters can be uncomfortable. Moreover, the transdermal devices are adapted for being used on skin. Sometimes, it is desirable to administrate a pharmaceutical substance locally, ie via mucous membranes. Plasters are not suitable for that kind of administration.

WO 99/47121 proposes administration of therapeutic agents by using absorbent products, such as tampons. However, nothing specific is disclosed regarding how the pharmaceutical substance is fixed to the absorbent product and about systems for facilitating up-take of the pharmaceutical substance into the body. Moreover, nothing is disclosed about which pharmaceutical substances that be used and which medical conditions that can be treated.

Accordingly, there is a need for improved devices for topic administration of pharmaceutical substances. There is also a large need for improved devices and methods for treating the premenstrual syndrome.

### SUMMARY OF THE INVENTION

It has now turned out that the above mentioned problems can be solved by providing an absorbent article, selected from the group consisting of a sanitary napkin, a panty liner, and an intralabial menstruation protection device, wherein the article is suitable for administrating a pharmaceutical substance. In one embodiment, the pharmaceutical substance is suitable for treating, mitigating and/or preventing the premenstrual syndrome. The article comprises an absorbent body and has a generally elongated shape. The article has a first surface which is intended to face the wearer, and it has a second surface which is intended to face away from the wearer. The first surface, which is intended to face the wearer, is at least partially covered with a transdermal therapeutic system containing a compound suitable for treating and/or preventing the pre-menstrual syndrome, whereby said compound is transdermally administrated to the wearer when said article is worn.

### DETAILED DESCRIPTION OF THE INVENTION

As disclosed herein, the term “pharmaceutical substance” relates to any pharmaceutical substance that is suitable for transdermal administration. Particularly interesting pharmaceutical substances in connection with the present invention are substances that are suitable for treating and/or preventing the pre-menstrual syndrome. Examples of substances that can be transdernally administrated and treat/prevent the pre-menstrual syndrome include linoleic acid and metabolites, such as gammalinolenic acid, dihomo-gamma linolenic acid, arachidonic acid, and adrenic acid, alpha-linolenic acid and metabolites, gestagens such as gestodene, levonogestrel, desogestrel, norethisterone, norethistosterone acetate and 3-keto-desogestrel, azaspirones or polycyclic amine derivatives including, among all, buspirone, gepirone, ipsapirone, and tandospirone, ergoline derivatives such as lisuride, bromolisuride, terguride, and proterguride.

As disclosed herein, the term “transdermal therapeutic system” relates to a composition or a device for transdermal administration of a pharmaceutical substance of interest. Accordingly, the transdermal therapeutic system can be a ceram, a lotion or an ointment for topical administration. Such ointments or creams are typically formulated as containing the active ingredient(s), generally in an amount ranging from about 0.001 to about 2% by weight. When formulated as an ointment, the active ingredients will typically be combined with either a paraffinic or a water-miscible ointment base. Alternatively, the active ingredients may be formulated in a cream with, for example, an oil-in-water cream base. Such topical formulations are well-known in the art and generally include additional ingredients to enhance the dermal penetration or stability of the active ingredients or the formulation. All such known topical formulations and ingredients are included within the scope of this invention.

The transdermal therapeutic system can also be a more complex solid system. Suitable such solid transdermal therapeutic systems are those usually employed for the percutaneous administration of active agents (Yie W. Chien: Transdermal Controlled Systemic Medications”, Marcel Dekker, Inc., New York and Basel, 1987; Cleary et al., “Transdermal Drug Delivery”, Cosmetics & Toiletries 106:97-109 (1991)). The transdermal therapeutic system is arranged on a surface of the absorbent article, which is intended to face the wearer. Typically, such a transdermal system comprises

a) optionally a backing layer;

b) one to three matrix layers that adhere to the cover layer and that contains a pharmaceutical substance, in particular a pharmaceutical substance that can be used to treat and/or prevent premenstrual syndrome, together with matrix-forming compounds and optionally penetration-enhancing agents; and optionally

c) a removable protective layer.

Solid transdermal therapeutic systems are typically manufactured by dissolving the active pharmaceutical substance and matrix-forming compounds, optionally together with penetration-enhancing agents, in a volatile solvent, thereby preparing a homogenous mixture. The backing layer is coated with this homogenous mixture, and the resulting laminate is dried. Finally, a triple laminate is formed by adding a removable protective layer.

Suitable matrix-forming compounds, or medicinally usual adhesives, are, for example, polyacrylates, silicones, polyurethanes, block polymers, styrene-butadiene copolymers as well as natural or synthetic rubbers, such as, e.g. polyisobutylenes. As additional matrix-formers, cellulose ether, polyvinyl compounds or silicates are to be considered. To increase the stickiness, the usual additives such as, for example, tackyfying resins and oils, can be added to the matrix obtained.

Suitable volatile solvents are, for example, lower alcohols, ketones or lower carboxylic acid esters, such as ethanol, isopropanol, acetone or ethyl acetate, polar ethers, such as tetrahydrofuran, lower hydrocarbons, such as cyclohexene or benzine, or else halogenated hydrocarbons, such as dichloromethane, trichloromethane, trichlorotrifluoroethane and trichlorofluoromethane. There is no need for an explaination that mixtures of these solvents are also suitable.

Suitable penetration-enhancing agents are, for example, monovalent or multivalent alcohols, such as ethanol, 1,2-propanediol or benzylalcohol, saturated and unsaturated fatty alcohols with 8 to 18 carbon atoms, such as lauryl alcohol or cetyl alcohol, hydrocarbons, such as mineral oil, saturated and unsaturated fatty acids with 8 to 18 carbon atoms, such as stearic acid or oleic acid, fatty acid esters with up to 24 carbon atoms or dicarboxylic acid diesters with up to 24 carbon atoms.

Fatty acid esters, which are suitable as penetration agents are, for example, those of acetic acid, caproic acid, lauric acid, myristic acid, stearic acid and palmitic acid, such as, for example, methyl ester, ethyl ester, propyl ester, isopropyl ester, butyl ester, sec-butyl ester, isobutyl ester, butyl ester, sec-butyl ester, isobutyl ester, tertbutyl ester or monoglycemic esters of these acids. Suitable dicarboxylic adiesters are, for example, diisopropyl adipate, diisobutyladipate and diisopropyl sebacate.

Other penetration-enhancing agents are phosphatide derivates, such as lecithin, terpenes, arnides, ketones, urea and its derivatives or ethers, such as, for example, dimethyl isosorbide and diethylene glycol monoethyl ether. There is no need for a more detailed explanation that also mixtures of these penetration-enhancing agents are suitable.

As protective layers, all films that are usually used in solid transdermal therapeutic systems are suitable. Such films are, for example, siliconized or fluoropolymercoated.

As a backing layer, for example, 10 to 100 μm thick films made of polyethylene or polyester can be used selectively pigmented or metallized in this system. The backing layer of the solid transdermal administration system is then bound to a surface of the absorbent article which is intended to face the wearer. Alternatively, the backing layer can be the surface of the absorbent article which is intended to face the wearer. The pharmaceutical sustance layer applied on it preferably has a thickness of 20 to 500 μm. The release of active ingredients usually takes place over a surface area of 1-10 cm².

The above-described components for topical and transdermal administration are merely representative. Other materials as well as processing techniques and the like are set forth in Part 8 of Remington's Pharmaceutical Sciences, 18th edition, 1990, Mack Publishing Company, Easton, Pa., 18042.

As already mentioned, the present invention relates to an absorbent article, selected from the group consisting of a sanitary napkin, a panty liner, and an intralabial menstruation protection, where the absorbent article also comprises a transdermal therapeutic system. Examples of such an absorbent article will now be described with reference to the enclosed drawings, in which:

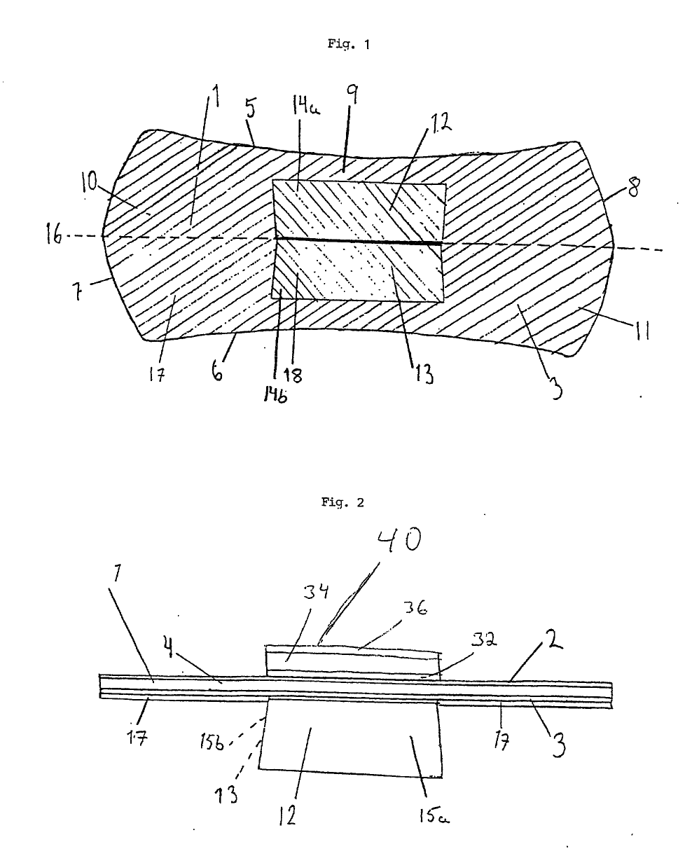

FIG. 1 illustrates a sanitary napkin from that side of the napkin which is intended to lie distal from the wearer;

FIG. 2 illustrates the napkin from one long side thereof. The transdermal therapeutic system is arraged on the side facing the wearer. The flaps of the napkin is disposed in a first use position;

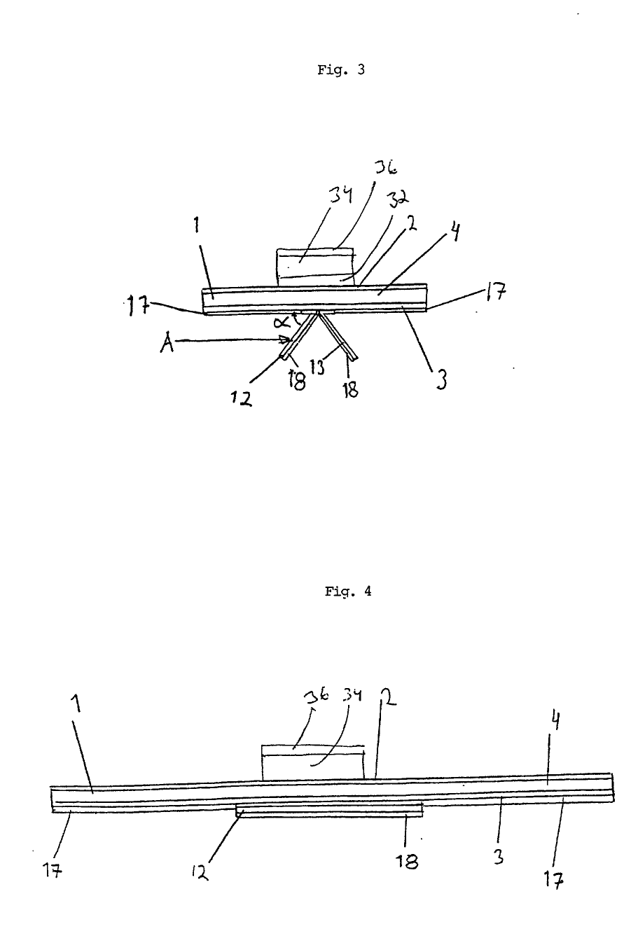

FIG. 3 illustrates the napkin, including a transdermal therapeutic system, from one short side thereof with flaps disposed in a first use position;

FIG. 4 illustrates a sanitary napkin, including a transdermal therapeutic system, from one long side thereof with flaps in a second use position;

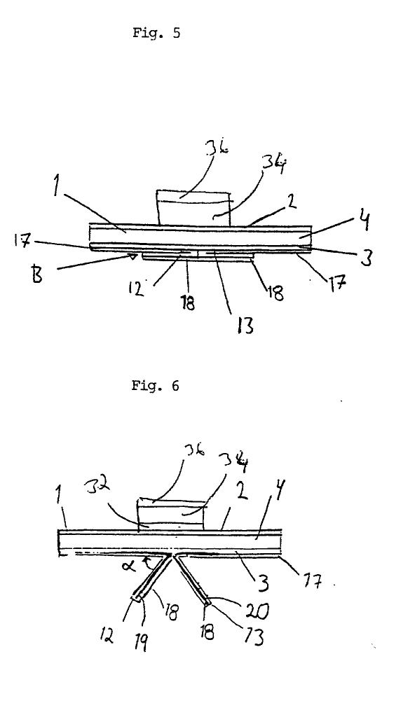

FIG. 5 illustrates a sanitarynapkin, including a transdermal therapeutic system, from one short side thereof with flaps disposed in a second use position;

FIG. 6 illustrates a sanitary napkin, including a transdermal therapeutic system, according to an alternative embodiment of the invention, and shows the napkin from one short side thereof with flaps disposed in a first use position;

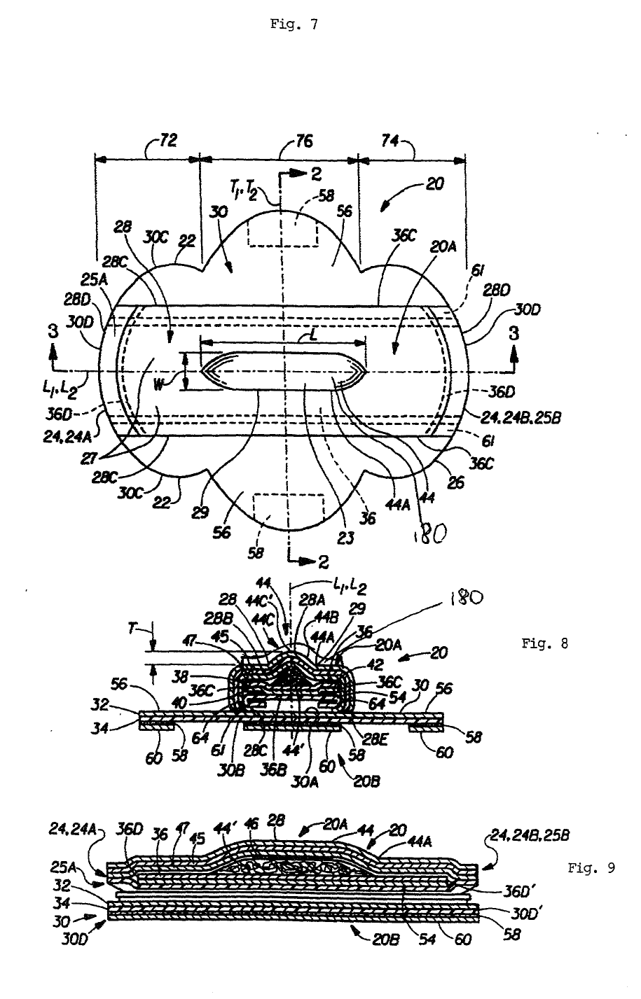

FIG. 7 is a top plan view of a preferred sanitary napkin embodiment of the present invention;

FIG. 8 is a sectional view of the preferred sanitary napkin embodiment shown in FIG. 7 taken along line 2-2 of FIG. 7;

FIG. 9 is a sectional view taken along line 3-3 of FIG. 7. The transdermal administration system is not shown;

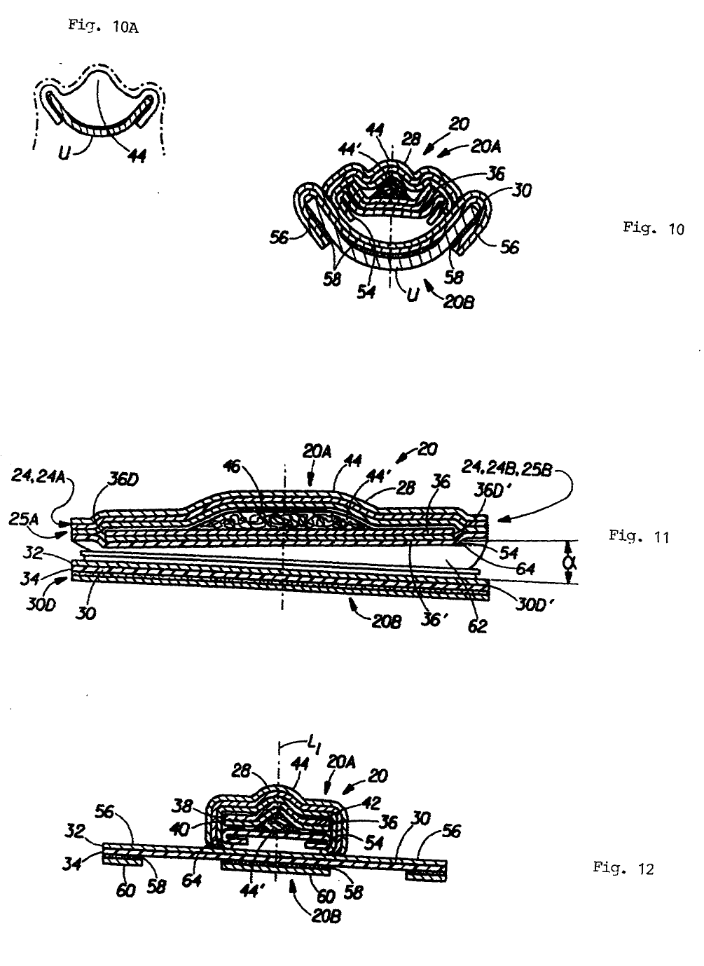

FIG. 10 is a sectional view taken from a similar angle to that of FIG. 8 which shows the separation of the absorbent core of the sanitary napkin from the backsheet. The transdermal administration system is not shown;

FIG. 10A is a simplified schematic view which shows how the sanitary napkin preferably fits adjacent the wearer's body. The transdermal administration system is not shown;

FIG. 11 is a sectional view taken from a similar angle to that of FIG. 9 which shows a side view of the separation of the absorbent core from the backsheet. The transdermal administration system is not shown;

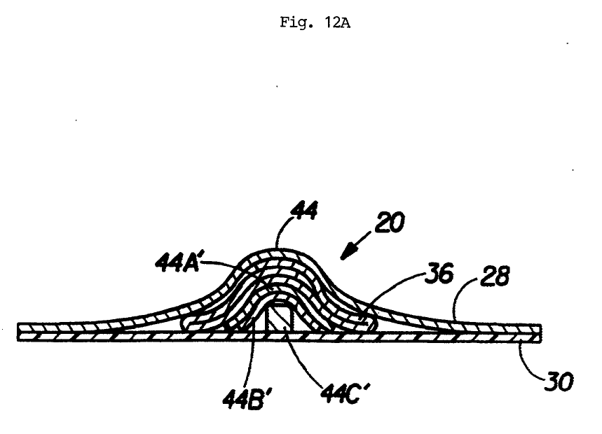

FIG. 12 is a sectional view of a sanitary napkin taken from a similar angle to that of the sanitary napkin of FIG. 8 which shows an alternative placement of the hump-forming element used in the sanitary napkin of the present invention. The transdermal administration system is not shown;

FIG. 12A is a sectional view of a sanitary napkin taken from a similar angle to that of the sanitary napkin of FIG. 8 which shows an alternative embodiment of the hump-forming element. The transdermal administration system is not shown;

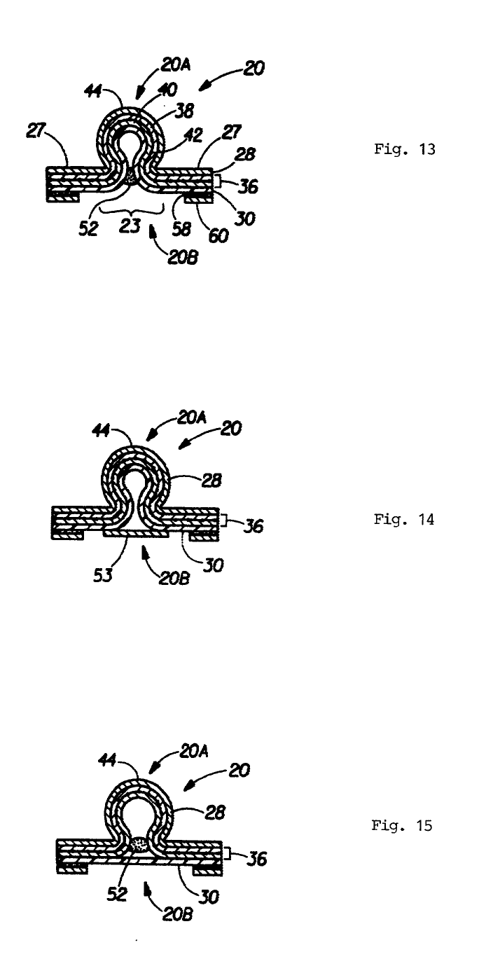

FIG. 13 is a sectional view of an alternate embodiment sanitary napkin taken at an angle similar to that of FIG. 8 in which the hump is created by laterally gathering portions of the central region of the sanitary napkin and securing them by adhesive. The transdermal administration system is not shown;

FIG. 14 is a sectional view of an alternate embodiment sanitary napkin taken at an angle similar to that of FIG. 8 in which the hump is created by laterally gathering portions of the central region of the sanitary napkin and securing them by a separate retaining means. The transdermal administration system is not shown;

FIG. 15 is a sectional view of an alternate embodiment sanitary napkin taken at an angle similar to that of FIG. 8 in which the hump is created by laterally gathering the topsheet and core only of the sanitary napkin. The transdermal administration system is not shown;

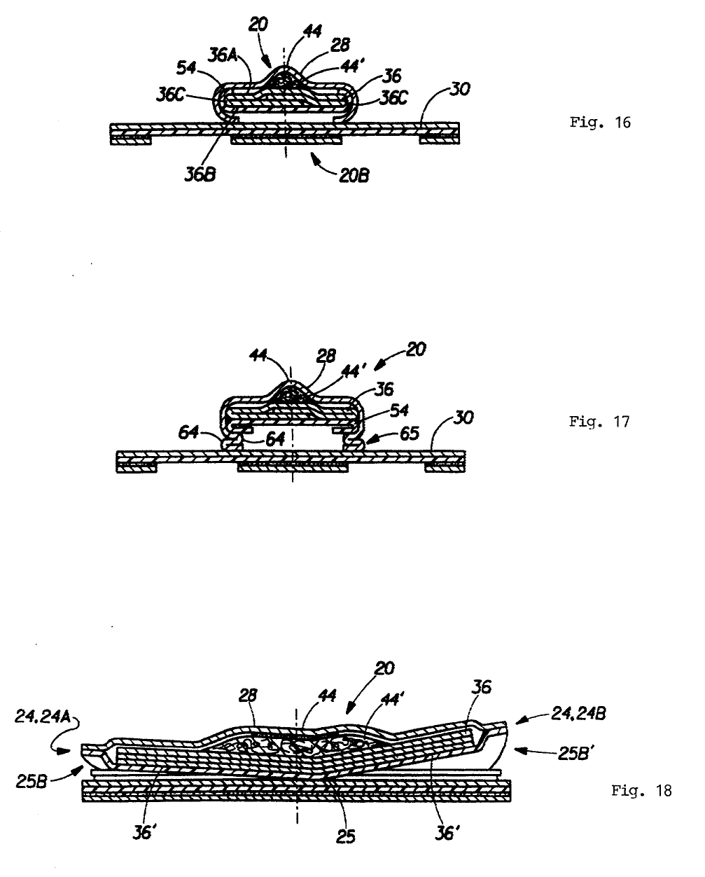

FIG. 16 shows an alternative version of the sanitary napkin of the present invention that has an optional interliner of an alternative configuration. The transdermal administration system is not shown;

FIG. 17 is a sectional view of an alternate embodiment sanitary napkin taken at an angle similar to that of FIG. 8 in which longitudinally disposed accordion style pleats control the amount of separation of the core from the backsheet. The transdermal administration system is not shown;

FIG. 18 is a sectional view of an alternate embodiment sanitary napkin taken at an angle similar to that of FIG. 9 in which the core is attached to the backsheet at a transverse juncture located between the end edges of the sanitary napkin. The transdermal administration system is not shown;

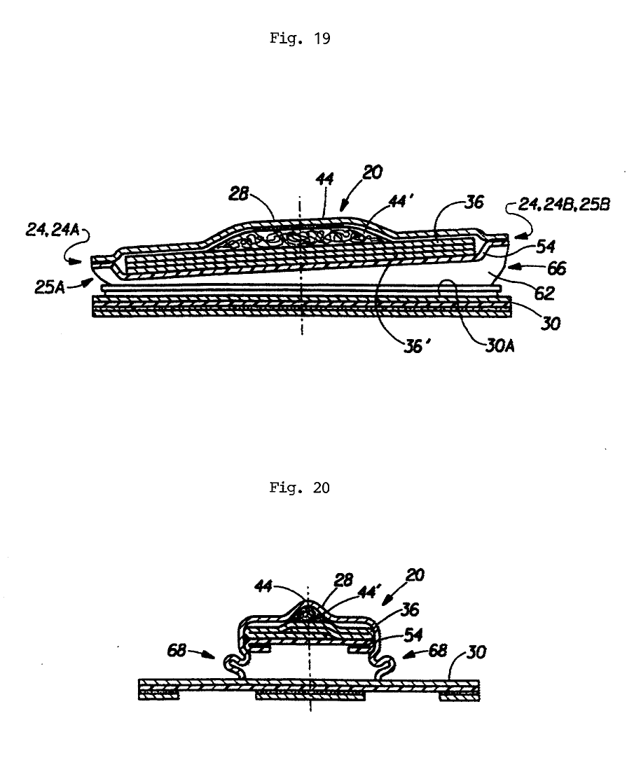

FIG. 19 is a sectional view of an alternate embodiment sanitary napkin taken at an angle similar to that of FIG. 9 in which the core is attached to the backsheet at a transverse juncture located at one end edge of the sanitary napkin and the opposing end edge of the core is attached to the backsheet by a material for controlling the separation therebetween. The transdermal administration system is not shown;

FIG. 20 is a sectional view of an alternate embodiment sanitary napkin taken at an angle similar to that of FIG. 8 in which the amount of separation of the core form the backsheet is controlled by a flaccid material. The transdermal administration system is not shown;

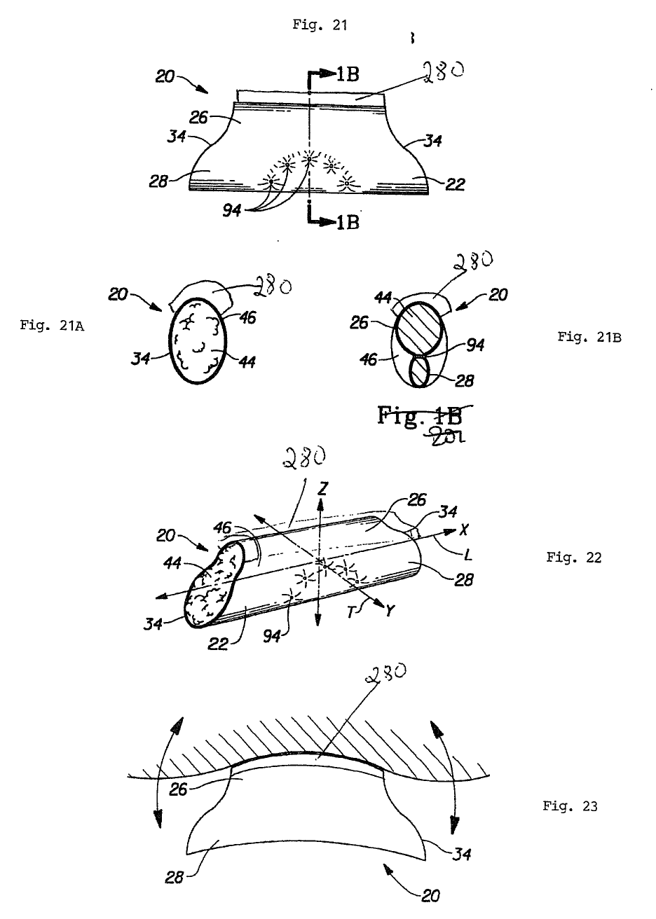

FIG. 21 is a side view of an absorbent interlabial device having a main absorbent portion with shaped ends;

FIG. 21A is an end view of the absorbent interlabial device shown in FIG. 21;

FIG. 21B is a cross sectional view taken through the transverse centerline of the absorbent interlabial device shown in FIG. 21 along line 1B-1B;

FIG. 22 is a perspective view of the absorbent interlabial device shown in FIG. 21;

FIG. 23 is a simplified schematic side view of the absorbent interlabial device of the present invention that is formed into a convex configuration when placed against a wearer's body;

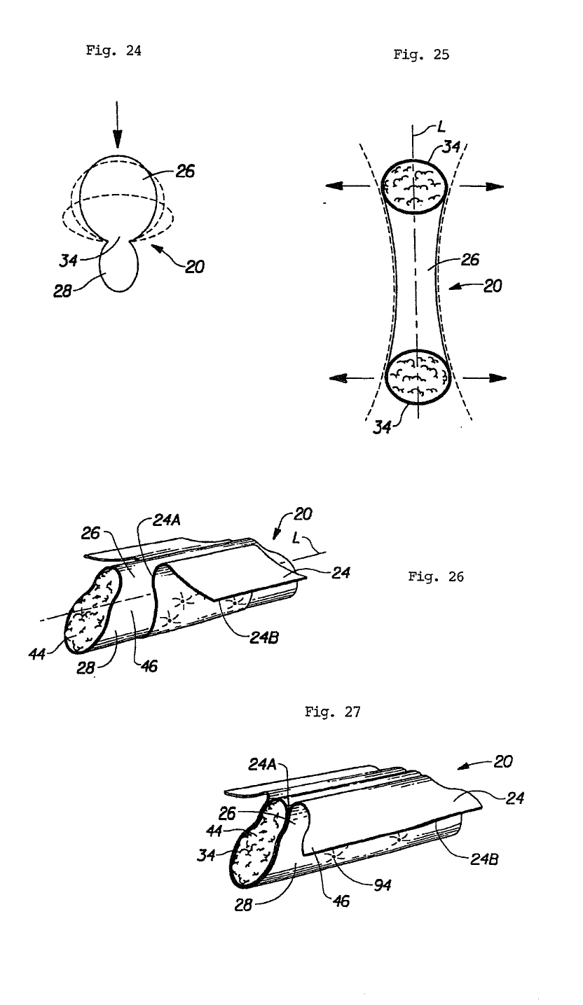

FIG. 24 is an end view of an absorbent interlabial device which shows how the ends flatten when the interlabial device is bent into the configuration shown in FIG. 23. The transdermal administration system is not shown;

FIG. 25 is a simplified schematic top view of the absorbent interlabial device which shows how the ends of the device widen to fit the configuration of the interlabial space. The transdermal administration system is not shown;

FIG. 26 is a perspective view of an interlabial device according to the present invention which has an optional pair of flexible extensions joined to the main absorbent portion. The transdermal administration system is not shown;

FIG. 27 is a side view of an interlabial device having an optional pair of flexible extensions that have end edges with the same configuration as the ends of the main absorbent portion. The transdermal administration system is not shown;

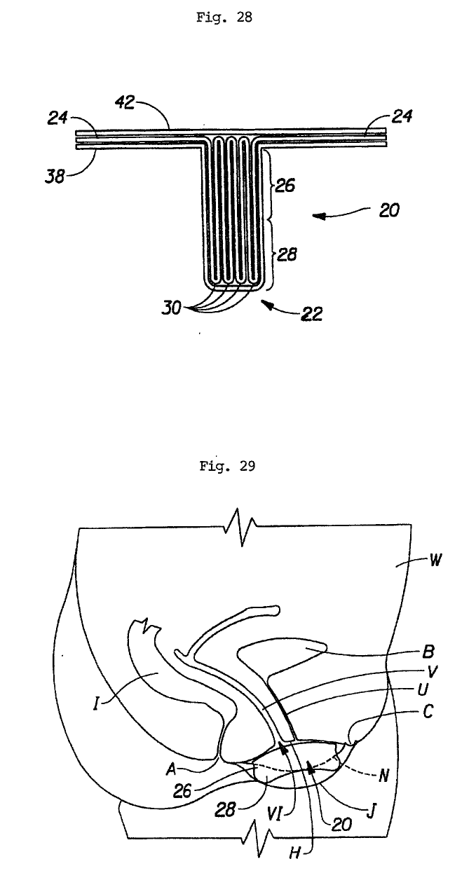

FIG. 28 is an end view of a preferred embodiment of the present invention having a pleated main absorbent portion. The transdermal administration system is not shown; and

FIG. 29 is a cross-sectional saggital view of a human female wearer showing the placement of the absorbent interlabial device in the wearer's interlabial space. The transdermal administration system is not shown;

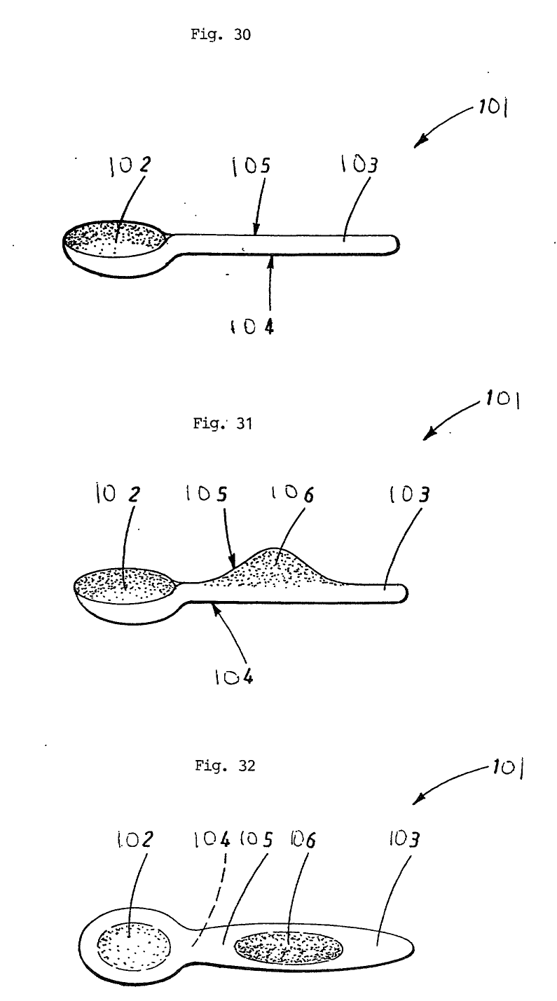

FIG. 30 is a side view of an interlabial device comprising a transdermal administration system according to another embodiment of the invention, before use;

FIG. 31 is a side view of the interlabial device in FIG. 30 during use after being wetted;

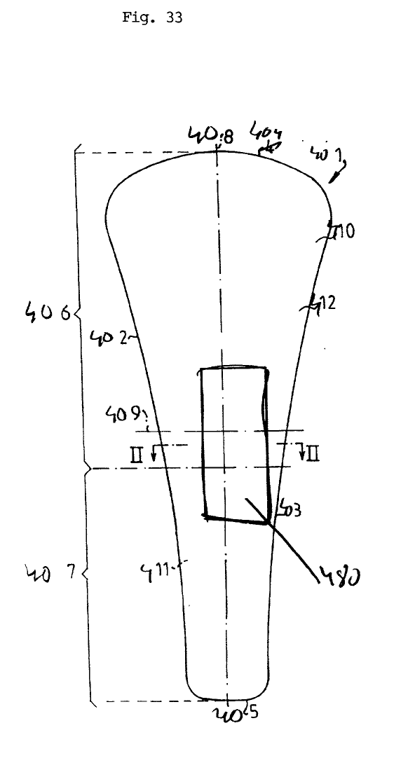

FIG. 32 is a view from above of the interlabial device in FIGS. 30-31 during use. The transdermal administration system is not shown;

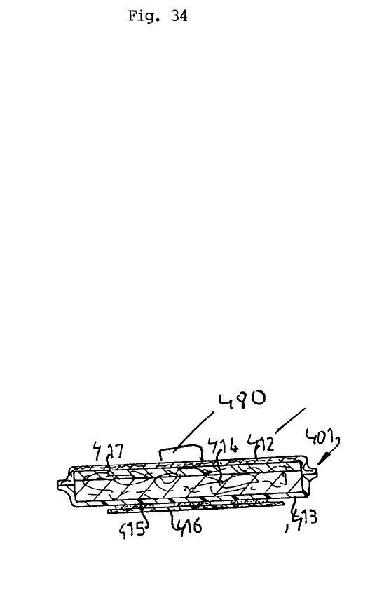

FIG. 33 shows a plan view of a sanitary napkin specially adapted for being used in string briefs; and

FIG. 34 shows a section along line II-II through the sanitary napkin in FIG. 33.

The sanitary napkin illustrated in FIGS. 1-6 includes a main body 1 that has a liquid-permeable sheet 2 on that side of the main body which lies proximal to the wearer in use, a liquid-impermeable sheet 3 on that side which is intended to lie distal from the wearer in use, and an absorbent layer 4 located between the aforesaid sheets. Finally, a transdermal therapeutic system 40 is located on the liquid-permeable sheet 2.

The main body 1 has an essentially oblong shape and is defined in the plane by two long sides 5, 6 and two short sides 7, 8. The sanitary napkin also includes a central part 9 disposed in the longitudinal direction of the article between its two end parts 10, 11. Each of the central part 9 and end parts 10, 11 take up approximately one-third of the length of the article.

The liquid-impermeable sheet or back sheet 3 is comprised of liquid-impervious material. Thin, liquid impervious plastic films are suitable for this purpose, although it is possible to use initially liquid permeable material that has been provided with a layer of plastic, resin or some other liquid impervious material. This prevents leakage of liquid from the backside of the absorbent article. The back sheet 3 may be comprised of any material that fulfils the liquid-impermeability criterion and is sufficiently flexible and skin-friendly for the purpose intended. Examples of materials suitable as sheets are plastic films, nonwoven and laminates thereof. The plastic film may be comprised of polyethylene, polypropylene or polyester. Alternatively, the back sheet may be comprised of a laminate of a liquid-impermeable plastic layer facing towards the absorbent body and a nonwoven layer facing towards the wearer's underpants. This construction provides a leakage-safe back sheet having a fabric-like feel.

The absorbent layer 4 is suitably comprised of cellulose pulp. The pulp may be in roll form, in bales or in sheets that have been dry-defibrated and converted to a fluffed state in a pulp mat, at times while admixing the pulp with so-called superabsorbents which, as before mentioned, are polymers that are capable of absorbing water or body fluid in quantities corresponding to several times their own weight. Examples of other materials that can be used in this context are different types of natural fibres, such as cotton fibres, peat moss or the like. Naturally, the use of absorbent synthetic fibres or mixtures of natural fibres and synthetic fibres is also conceivable. The absorbent material may also contain other components, such as shape-stabilizing agents, liquid dispersing agents or binding agents, such as thermoplastic fibres that have been heat-treated to hold together short fibres and particles in a coherent unit. It is also known to use different types of absorbent foam material in the absorbent body.

The absorbent layer 4 of the sanitary napkin is covered by a liquid-permeable top sheet 2 provided on that side of the main body 1 which faces towards the wearer in use. The top sheet 2 may be comprised of perforated plastic film, plastic or textile net, nonwoven material or a laminate comprised of a plastic layer and a nonwoven layer, for instance. The plastic may be a thermoplastic, such as polyethylene. The nonwoven material may be comprised of natural fibres, such as cellulose or cotton, or synthetic fibres, such as polyethylene, polypropylene, polyester, polyurethane, nylon or regenerated cellulose fibres.

The main purpose of the casing sheet 2 of the sanitary napkin is to guide fluid into the absorbent layer 4, to be soft and comfortable against the wearer's body, and to prevent so-called rewetting, i.e. the return of absorbed body fluid onto the wearer's skin. With regard to comfort and the avoidance of skin irritations, it is important that the surface of that part of the napkin which lies against the wearer's skin remains as dry as possible in use. A dry napkin surface is also felt to be more cool and comfortable by the user, and is more attractive than a soiled, wet surface when changing the napkin, both from a purely visual aspect and from a handling aspect.

The article also includes two fastener flaps 12, 13, each having an inside 14a, b and an outside 15a, b for fastening the article to the wearer's underclothes. The flaps 12, 13 are fastened to the main body 1 of the napkin, for instance glued, welded or sewn, on that side of the body which presents the fluid impermeable sheet 3. The flaps are fastened to the fluid impermeable sheet 3 of the main body at a distance from the long sides 5, 6 of the article. The flaps are preferably fastened in the center of the rear side of the article, as seen from the short sides 7, 8 thereof, although the flaps may alternatively be fastened closer to the long sides of said article while maintaining a good fastener function in the V-shaped crotch part of underwear. When the width of the article in that part where the flaps or wings are fastened, i.e. the central part 9, is 5 cm, the preferred distance from the long sides 5, 6 of the article will be about 2.5 cm. Naturally, these relationships are to scale: For instance, if the width of the central part is 8 cm, then the preferred distance from the long sides of the article will be 4 cm. In the illustrated case, the flaps 12, 13 are fastened adjacent one another on the fluid or liquid impermeable sheet 3. Alternatively, the flaps 12, 13 may be fastened closer to the long sides 5, 6 of the article and not positioned adjacent one another. For instance, when the width of the article is 5 cm, the flaps 12, 13 may be fastened at a distance of 1.5 cm from each long side 5, 6. When the width of the article is 8 cm, the flaps 12, 13 may be fastened at a distance of 3 cm from the long sides of the article.

As already mentioned, the absorbent article includes a transdermal therapeutic system 40 which is arranged on the liquid-permeable sheet 2. The active part 34 of the transdernal therapeutic system is either a cream, lotion or ointment, or alternatively a solid transdermal therapeutic system. The transdermal therapeutic system 40 is either arranged directly on the liquid-permeable sheet 2 and adhered using any suitable means. Alternatively, the transdermal therapeutic system may comprise a backing layer 32, which in turn is bound to the liquid-permeable sheet using any suitable means, such as a glue. The transdermal therapeutic system 40 may also be covered by a removable protective layer 36.

FIGS. 1-3 illustrate an article having fastener flaps 12, 13 disposed in a first use position A in which they define an angle α with the liquid-impermeable sheet 3. When the sanitary napkin is in an initial state of its application, the first use position A can be described as a position in which the flaps 12, 13 extend obliquely outwards from the back sheet 3 in a direction away from the longitudinal symmetry line 16 of the napkin and towards respective long sides 5, 6, such as to define an angle α. In other words, the angle α is defined between the extension direction of respective fastener flaps 12, 13 and the napkin back sheet. FIGS. 4-5 illustrate a sanitary napkin with the fastener flaps 12, 13 disposed in a second use position B. In use position B, the fastener flaps, or wings, lie against the back sheet 3 without defining an angle thereto. The first use position A shown in FIGS. 1-3 is suitable for mounting the sanitary napkin in boxer shorts, panty hose and similar types of underwear that have a broad, V-shaped crotch. The second use position B shown in FIGS. 4-5, is suitable for mounting the sanitary napkin in underwear that have a generally flat crotch part. The first use position A makes an angle α between the flaps 12, 13 and the liquid-impermeable sheet 3, this angle depending on the steepness of the V-shape of the crotch part. The angle α will vary with the body attitude of the wearer and his/her movements thereof and also according to the nature of the underwear in which the article is mounted. The angle α will vary between 0 and 90°. The angle will be relatively large, about 45-90° when the sanitary napkin is mounted in boxer shorts made of loose, flexible material, whereas when the napkin is mounted in panty hose, the angle will be smaller and in the region of 0-45°. Naturally, this data is approximate with regard to the position of the flaps 12, 13 in relation to the liquid-impermeable sheet 3 and thereby to the main body 1. The angle α will also vary in dependence on the movements and body attitudes of the wearer. However, the flaps will be resilient or elastic around the use position.

The first use position A primarily places special requirements on the article. In order to ensure that the main body 1 will not wrinkle or gather together when in use, the main body must be rigid or stiff. This is achieved by using one or more stiff layer, for instance a stiff liquid-permeable sheet 2, a stiff liquid impermeable sheet 3 or a stiff absorbent layer 4 in the main body 1, or by including stiff layers in the main body 1. One simple method of obtaining a stiff main body 1 which will not wrinkle in use is to form the absorbent layer 4 from a stiff material. A suitable material with regard to the absorbent layer 4 is the absorbent material described in WO 94/10956. This material is a dry-formed fibre sheet of high density and stiffness that can be used directly in an absorbent article without first being defibrated. A similar material having particularly suitable blood-absorption properties is described in WO 94/10953. Both of these materials have sufficient stiffness and ability to withstand deformation and therewith impart to the main body 1 the shape stability required by the present invention.

Rigid absorbent material of the aforesaid nature may be comprised of dry-formed sheet containing 5-100% cellulose fibre and having a density of between 0.2 and 1.0 g/cm³ and a weight by surface area of between 30 and 2000 g/m², wherein the sheet is formed by compressing a web that contains cellulose fibres without subsequent defibration and fluff formation. Since this material absorbs liquid relatively slowly, it may be appropriate to include in the absorbent layer 4 additional absorbent material which will absorb more rapidly, this material being placed nearest the liquid-permeable sheet 2. Air-laid material is an example of such rapid absorbent material.

It may be beneficial for the fastener flaps 12, 13 to be resilient in their first use position A. The resilience of the flaps 12, 13 around their use position A may also be beneficial when the sanitary napkin is used with the flaps 12, 13 in their second use position B. If the flaps 12, 13 are not fastened to the back sheet 3, the resiliency will cause the main body 1 to be pressed resiliently against the wearer's body therewith reducing the risk of the napkin gaping away from the wearer's skin and resulting in a more leakage-proof sanitary napkin. An elastic effect can be obtained by providing the main body 1 and the flaps 12, 13 with elastic threads 19, 20, which are mounted in a stretched state. One such construction is shown in FIG. 6. In this case, it may be convenient to cover the whole of the back sheet 3 with an area of pressure-sensitive adhesive 17. This will enable the flaps 12, 13 to be fastened to the back sheet 3 and the article to be used in its second use position B. If the user wishes to use the article with its flaps 12, 13 in their first use position A, the flaps 12, 13 are released from the back sheet 3 and spring back around to the first use position A. Alternatively, the outer surfaces 15a, b of the fastener flaps may be provided with a pressure-sensitive adhesive, instead of covering the whole of the back sheet 3 with said adhesive area 17.

The fastener means 17, 18 are comprised of self-adhesive surfaces, although other types of fasteners may be used, such as hook-and-loop fasteners or other types of fastener which can be fastened directly to the materials from which panties or underpants are normally made.

FIGS. 1-5 illustrate an example of how the fastener means can be applied. The fastener means 17, 18 are comprised of pressure-sensitive adhesive applied to the liquid-impermeable sheet 3 and on the inner surface 14a, b of the fastener flaps 12, 13. The fastener means are shown hatched in FIG. 1. The adhesive surface 17 applied to the liquid-impermeable sheet is used solely to fasten the article when said article is to be placed in underwear that have a substantially flat crotch region, in other words when the flaps 12, 13 are in their second use position B. In this case, the adhesive surface 18 on the inner surfaces 14a, b of the flaps is also used to fasten the article to the wearer's underwear. When the article is to be secured in underwear that have a V-shaped crotch region, such as boxer shorts, only the adhesive surface 18 on the inner surfaces 14a, b of the flaps is used. Naturally, it is not necessary to cover the whole of the liquid-impermeable sheet 3 with adhesive and it is conceivable to apply adhesive solely to the flaps 12, 13, i.e. the adhesive surface 18.

Although not shown in the drawings, the fastener means 17, 18 are covered with releasable protective paper prior to use, this paper being removed from the fastener means 17, 18 before use. The protective paper or like means may be omitted when the fastener means 17, 18 are comprised of a material which will not adhere spontaneously to other surfaces or when the fastening capacity is not impaired after being released from such spontaneous adherence to another surface.

The present invention also relates to a sanitary napkin or a panty liner provided with a central hump covered with a transdermal administration system. FIGS. 7-20 show such a sanitary napkins. A sanitary napkin 20 provided with a longitudinally-oriented elongated hump 144 covered with a transdermal administration system 180 on the body surface 120A of the sanitary napkin 120 is shown in FIGS. 7-8. In order to increase simplicity, the transdermal administration system is not shown in FIGS. 9-10.

The hump 144 is particularly useful in fitting into the space between the wearer's labial tissue. The hump 144 is intended to remain in contact with the surfaces of the wearer's labia majora adjacent the space between the labial tissue to more readily intercept menses and other bodily discharges when they leave the wearer's body. The hump 144 may be of such a size that it also at least partially fits against or in the wearer's perineum. Since the hump 144 will be in close proximity to the wearer's body, it should preferably be relatively soft. The hump 144 should preferably be capable forming a good fit and conforming to the shape of the space defined by the inwardly-facing surfaces of the wearer's labia majora.

The hump 144 may be at least partially comprised of (that is, it may be formed by) portions of one or more of the components of the sanitary napkin 120. The hump 144 can be formed by portions of one or more of any of the following components: the topsheet 128, the absorbent core 136, the backsheet 130, or any additional layers described herein that lie between the topsheet 128 and the backsbeet 130. Alternatively, or additionally, the hump 144 can be at least partially formed by a hump-forming element such as that designated 144′ in the drawings.

The hump 144 is preferably at least partially absorbent. The hump 144 is considered to be “absorbent”, for the purposes of the present invention, if any portion of the hump 144 is absorbent. It is not necessary that all portions of the hump 144 be absorbent, however. For instance, the hump 144 will be considered to be “absorbent” even when the topsheet 128 comprises a formed film that is not inherently absorbent itself, as long as one of the underlying components that forms the hump 144 is absorbent.

The hump 144 is “longitudinally-oriented” and “elongated”. The terms “longitudinal” and “longitudinally-oriented”, as used herein, mean that the hump 144 is oriented so that its largest dimension is oriented in the longitudinal direction. The term “elongated”, as used herein, means that the hump 144 is long in proportion to its width. The hump 144 has its own longitudinal and transverse centerlines L₂ and T₂ (shown in FIG. 7). (When the hump-forming element 144′ defines the shape of the hump 144, the hump-forming element 144′ will generally also have longitudinal and transverse centerlines of its own. These are typically the same as those of the hump 144.)

The location or position of the hump 144 is shown in FIG. 7. The hump 144 is centered on top of the body surface 120A of the sanitary napkin 120. In such a case, the longitudinal and transverse centerlines of the hump L₂ and T₂ coincide with the principal longitudinal and transverse centerlines L₁ and T₁ of the sanitary napkin 120. The hump 144 may alternatively be positioned in other embodiments so that it is offset from either the principal longitudinal or transverse centerlines of the sanitary napkin 120. Preferably, the hump 144 is at least generally centered about the principal longitudinal centerline L₁ of the sanitary napkin 120. (That is, the hump 144 is central or “medial”, or midway between the longitudinal edges 122 of the sanitary napkin 120). This enables the hump 144 to be positioned into the space between the wearer's labial tissue. The hump 144, however, does not have to be centered relative to the principal transverse centerline T₁ of the sanitary napkin 120. The hump 144 may, for instance, be offset from the principal transverse centerline when the sanitary napkin is asymmetrical about the principal transverse centerline. Preferably, however, at least part of the hump 144 is sufficiently centered relative to the principal transverse centerline that part of the hump 144 is located in the central region 176 of the sanitary napkin 120.

The hump 144 can be of any shape provided it at least partially fits into the space between the wearer's labial tissue. The hump 144 can be either symmetrical or asymmetrical about its longitudinal and transverse centerlines L₂ and T₂. In the preferred embodiment shown in FIGS. 7-9, the hump 144 is symmetrical about both. (When discussing the shape of the hump 144, it should be understood that when a hump-forming element 144′ is used, the shape of the absorbent hump 144 will generally be similar to that of the hump-forming element 144′. There are situations when the shapes of the hump 144 and the hump-forming element 144′ will differ somewhat, however. As one example, when the hump-forming element 144′ is cylindrical, because certain components of the sanitary napkin 120 will be draped over the hump-forming element 144′, the hump 144 may only take the shape of the upper portion of the cylindrical shape.) Some suitable shapes are described below.

The plan view shape of the hump 144 (that is, the shape of the absorbent hump 144 when viewed from directly above when the sanitary napkin 120 is in its flat, laid out condition) is shown in FIG. 7. The plan view shape of the hump 144 can be cigar-shaped (shown in FIG. 7), rectangular, oval, or some other suitable shape.

The transverse cross-sectional shape of the hump 144 is shown in FIG. 8. The hump 144 has sides which are slightly concave inward which taper to a rounded top surface. The hump 144 could, alternatively, be “box-shaped” so that it has a generally rectangular transverse cross-section. In still other alternatives, the hump 144 could have a cross-section that is semi-circular (for instance, if the bump-forming element 144′ is cylindrical). Alternatively, the transverse cross-sectional shape could be parabolic, triangular, in the shape of a modified rectangle that has straight side edges and a rounded top, or any other suitable shape. In still other alternatives, (as shown in FIGS. 13-15) the sides of the hump 144 could be concave outward.

The longitudinal cross-section of the absorbent hump 144 can also take various different shapes. These include, but are not limited to shapes in which the hump 144 is generally rectangular is longitudinal cross-section (for instance, if the hump-forming element 144′ is rectangular or cylindrical), and those in which the hump 144 is tapered. FIG. 9 provides an example of a hump 144 which is tapered from its transverse centerline T₂ toward both ends of the hump 144.

The hump 144 has a longitudinal dimension (length) L, a transverse dimension (width) W, and a vertical or z-direction dimension (“thickness” or “height”) T. (These are shown in FIGS. 7 and 8.) The length L of the hump 144 is measured longitudinally from the most outboard point at the base 144A of the hump on one end of the hump to a similar point on the other end of the hump 144. The width W of the hump 144 is generally measured transversely from the most outboard point on one side of the hump 144 to a similar point on the other side of the hump 144.

The portions of the sanitary napkin at the base 144A of the hump used for the foregoing measurements are located at those places where there is a visible change in direction or degree of curvature or height in the plane defined by the surrounding regions 127. It should also be understood that if the transverse cross-sectional shape of the hump 144 is tapered, the width of the hump 144 may vary between the top of the hump to the base of the hump 144. The hump 144 may, therefore, have different widths (and other dimensions) depending on the portion of the hump 144 that is measured. For instance, the width can be measured using two points which lie in the plane of the base 144A of the hump. Alternatively, the width can be measured in any other plane which cuts through the hump 144 and is parallel to the plane of the base of the hump 144.

The height T of the hump 144 is the perpendicular distance from the top surface of the topsheet 128 at the base 144A of the hump 144 to the point of maximum amplitude 144C′ on the top 144C of the hump 144. The caliper of the hump 144 also includes the thickness of portions of the sanitary napkin which lie under the hump. (Thus, the height T of the hump 144 differs from the caliper of the hump 144. The caliper will typically be used to express the entire thickness of the sanitary napkin at the hump because it is easiest to measure.)

The dimensions of the hump 144 can vary between certain limits. The preferred dimensions of the hump 144 are provided below. In some cases, these dimensions may be expressed in terms of the dimensions of the hump-forming element 144′. This may be done because it may be easier to measure the dimensions of such an element. Any of the following dimensions which are expressed in terms of the dimensions of the exterior dimensions of the hump 144 may, thus, also be used as approximations of the dimensions of the hump-forming element 144′ and vice versa. If a hump-forming element 144′ is used, however, the exterior dimensions of the hump 144 will generally be slightly greater than the dimensions of the hump-forming element 144′. The exterior dimensions of the hump 144 differ from those of the hump-forming element 144′ by the thickness of the components of the sanitary napkin that overlay the hump-forming element 144′ and by any additional thicknesses created when these components are positioned or “draped” over the hump-forming element 144′.

The length L of the hump 144 can range from between about 2 cm to the length of the absorbent core 136. (The length of the absorbent core 36 can, for example, be about 20 cm to about 22 cm.). Preferably, the length L of the hump 144 is between about 4 cm and about 15 cm, and more preferably, is between about 4 cm and about 10 cm, and most preferably is between about 4 cm and about 7.5 cm. With regard to the length of the hump 144, it should be noted that it is believed that the sanitary napkin 120 will perform better when the length of the hump is shorter than some of the lengths specified above at the high ends of the ranges. The preferred lengths are in the range of between about 2 or 4 cm to about 10 cm. More preferred lengths are between about 4 cm to about 10 cm. These typically work better because the hump will fit more completely in the space between the wearer's labial tissue and in the perineal groove. The embodiments having humps 144 longer than these lengths, while still operable, are less preferred because they represent further departures from structures which fit entirely within the space between the wearer's labial tissue and perineal groove.

The width W of the hump 144 may be as great as between about 0.5 cm and about 5 cm, and is more preferably between about 1 cm to about 5 cm, and more preferably still is between about 1 cm and about 4.5 cm at the base, and most preferably is between about 1 cm and about 4 cm. The dimensions of the hump 144 will often decrease from the base 144A of the hump to the top 144C of the hump 144. When the hump 144 tapers from the base 144A to the top 144C as shown in FIG. 8, the top 144C of the hump 144 may resemble the top of a ridge and have a very small width at the point of maximum amplitude 144C′. The dimensions of the portions of the hump 144 above the base 144A, particularly (the width of) those portions that comprise the upper half of the hump 144, may more closely approximate the dimensions of the hump-forming element 144 (described below) than the dimensions of the lower half of the hump. (The “upper half” of the hump refers to those portions spaced greater than or equal to ½ T from the base.) That is because these portions of the hump 144 are more likely to be the portions of the hump that at least partially fit into the space between the wearer's labia. In still other alternative embodiments, the size of the base of the hump 144 could also be within the ranges of dimensions given below for the hump-forming element 144′.

The caliper of the portions of the sanitary napkin 120 containing the hump 144 can be measured at different portions of the hump 144. Preferably, the caliper of the hump 144 is measured at the point of maximum amplitude. The caliper of the point of maximum amplitude 144C′ of the hump 144 is greater than that of the surrounding regions 127. The caliper of the sanitary napkin 120 at the point of maximum amplitude 144C′ of the hump 144 is preferably greater than about 2 times the caliper of the surrounding regions 127 of the sanitary napkin. (The point of maximum amplitude 144C′ is typically located along the longitudinal centerline of the hump 144.) The sanitary napkin 120 preferably has a caliper at the point of maximum amplitude of the hump of at greater than about 3 millimeters, more preferably between about 4 millimeters and about 15 millimeters. The thickness T of the hump 144 can be calculated by subtracting the caliper of the surrounding regions 127 at the base 144A of the hump 144 from the caliper of the hump.

The sanitary napkin (as discussed above) may have a hump 144 that is at least partially formed by a hump-forming element 144′. The hump-forming element 144′ not only partially forms the hump 144, it may also provide extra absorbency in the target region of the sanitary napkin 120 (the region where menses and other bodily discharges are typically deposited).

The hump-forming element 144′ shown in FIGS. 7-9 is positioned on top of (i.e., above) the absorbent core 136 and under an optional wipe acquisition sheet 145 and wet-laid tissue 147 (described in greater detail below). In alternative embodiments, the hump-forming element 144′ can be positioned between nearly any of the other components or layers described herein. For instance, the hump-forming element 144′ can be positioned between one of the tissue layers (such as either 138 or 140) that comprises the absorbent core 136 and the layer of superabsorbent material particles 142 in the core. In such an embodiment, the hump-forming element 144′ may be considered to be positioned within the absorbent core 136. The hump forming element 144′ also may be positioned adjacent to one of the faces of the absorbent core 136. The hump-forming element 144′ can be integral with the absorbent core 136 (i.e., it can be a part of the absorbent core 136), or it can be a separate element from the absorbent core 136. In still other embodiments (such as shown in FIG. 12), the hump-forming element 144′ may be positioned below the absorbent core 136. In still other alternative embodiments, the hump-forming element 144′ can comprise a longitudinally-oriented tube that is attached to the body surface of the sanitary napkin.

The hump-forming element 144′ will normally be a lofted (i.e., relatively thick) component. The hump-forming element 144′ should also preferably be soft and non-irritating to the wearer since it may be placed in relatively close contact with the wearer's body. The hump-forming element 144′ may also be somewhat flexible. However, it is not necessary for the hump-forming element 144′ to be as flexible as the surrounding regions 127 of the sanitary napkin.

The hump-forming element 144′ may also be absorbent. The hump-forming element 144′, however, need not be absorbent if it is placed under the absorbent core 136.

The hump-forming element 144′ preferably should be held in place in the sanitary napkin 120 so that it is prevented from shifting a great deal longitudinally or laterally when the sanitary napkin 120 is worn. The hump-forming element 144′ may have some lateral mobility to adjust to non-symmetric anatomy and misplacement of the sanitary napkin in the panty by the wearer. The hump-forming element 144′ does not have to be secured to any other component of the sanitary napkin 120, however. The hump-forming element 144′ could, for instance, be merely fit snuggly between components of the sanitary napkin or within a component of the sanitary napkin. Typically, however, the hump-forming element 144′ is secured to the components that lie both above and below it.

The hump-forming element 144′ can be secured to these components with any suitable hump-forming element securement means 146, such as an adhesive. The hump-forming element securement means 146 may coincide in area with the area of all the upper and lower surfaces of the hump-forming element 144′ (or it may only coincide with a portion of these surfaces). The hump-forming element securement means 146 should not inhibit flow of menses and other exudates to the hump-forming element 144′ or to the core 136. If the hump-forming element securement means 146 is an adhesive, the path of flow to the core 136 can be preserved in several ways. These include spreading the adhesive sufficiently thin so that a substantial number of the apertures in the topsheet material are not covered with adhesive. Many types of adhesives are suitable for use as the hump-forming element securement means 146, including water-based adhesives and hot melt adhesives.

The dimensions of the hump-forming element 144′ are as follows. The length of the hump-forming element 144′ is preferably within approximately the same ranges as the ranges set forth above for the length of the hump 144. The width of the hump-forming element 144′ may be between about 0.5 cm and about 5 cm. The thickness of the hump-forming element 144′ may be between about 0.3 cm up to about 1 cm or about 1.3 cm.

In another embodiment, the hump-forming element 144′ could comprise several separate components which are stacked on top of each other. For instance, as shown in FIG. 106A, the hump-forming element 144′ comprises several distinct strips that are positioned under a thin absorbent core 136. The hump-forming element preferably comprises an upper component such as a relatively thin first strip 144A′. The thin strip 144A′ is placed on top of a lower component such as a relatively thick, but narrower second strip 144B′. The second strip 144B′ is placed on top of a still narrower third strip 144C′. The relatively thick second and third strips serve as a fulcrum around which the core and first strip are bent. The components of the hump-forming element 144′ may be made of different materials. In one version of this embodiment, the upper components may be comprised of an absorbent material and the lower components may be non-absorbent, or vice versa. In still other versions, the separate components of this embodiment could be diposed above, within, or partially above and/or within and/or below, the absorbent core. It should be understood that all the various possible arrangements of such separate components are within the scope of the present invention.

The material used for the hump-forming element 144′ can be any of those materials specified for use in the absorbent core 136. Preferably, however, the hump-forming element 144′ comprises a material that is more resilient than an airfelt mass. One suitable material for use in the hump-forming element 144′ is a mass of chemically modified, cross-linked cellulosic fibers such as those described in U.S. Pat. No. 4,888,093. Another suitable material is a tuft of superabsorbent fibers, such as those formerly manufactured by Arco Chemical Company of Newton Square, Pa. under the trade-mark FIBERSORB and those currently manufactured by Courtaulds, Ltd., West Midlands, England. Superabsorbent fibers are discussed more fully in U.S. Pat. No. 4,855,179. Still other suitable materials for the hump-forming element 144′ are the capillary channel fibers (fibers having channels on their exterior surfaces) described in greater detail in EP 391,814.

The present invention is also directed to an absorbent interlabial device comprising a transdernal administration system. FIG. 21 shows one embodiment of such an absorbent interlabial device, interlabial device 220. The present invention, however, is not limited to a structure having the particular configuration shown in the drawings.

As used herein the term “absorbent interlabial device” refers to a structure which has at least some absorbent components, and which is specifically configured to reside at least partially within the interlabial space of a female wearer during use. Preferably, more than half of the entire absorbent interlabial device 220 of the present invention resides within such interlabial space, more preferably substantially the entire absorbent interlabial device 220 resides within such interlabial space, and most preferably the entire absorbent interlabial device 220 resides within such interlabial space of a female wearer during use.

As used herein, the term “interlabial space” refers to that space in the pudendal region of the female anatomy which is located between the inside surfaces of the labia majora extending into the vestibule. Located within this interlabial space are the labia minor, the vestibule and the principal urogenital members including the clitoris, the orifice of the urethra, and the orifice of the vagina. Standard medical authorities teach that the vestibule refers to the space bounded laterally by the inside surfaces of the labia minora and extending interiorly to the floor between the clitoris and the orifice of the vagina. Therefore, it will be recognized that the interlabial space as defined above may refer to the space between the inside surfaces of the labia majora, including the space between the inside surfaces of the labia minora also known as the vestibule. The interlabial space for purposes of the present description does not extend substantially beyond the orifice of the vagina into the vaginal interior.

The term “labia” as used herein refers generally to both the labia majora and labia minora. The labia terminate anteriorly and posteriorly at the anterior commissure and the posterior commissure, respectively. It will be recognized by those skilled in the art that there is a wide range of variation among women with respect to the relative size and shape of labia majora and labia minora. For purposes of the present description, however, such differences need not be specifically addressed. It will be recognized that the disposition of the absorbent interlabial device into the interlabial space of a wearer as defined above will require placement between the inside surfaces of the labia majora without regard to the precise location of the boundary between the labia majora and the labia minora for a particular wearer. For a more detailed description of this portion of the female anatomy, attention is directed to Gray's Anatomy, Running Press 1901 Ed. (1974), at 1025-1027.

The absorbent interlabial device 220 shown in FIGS. 21-22 has a longitudinal centerline L which runs along the “x” axis shown in FIG. 22. The term “longitudinal”, as used herein, refers to a line, axis or direction in the plane of the interlabial device 220 that is generally aligned with (e.g., approximately parallel to) a vertical plane which bisects a standing wearer into left and right body halves when the interlabial device 220 is worn. The terms “transverse,” “lateral,” or “y direction” as used herein, are interchangeable, and refer to a line axis or direction that is generally perpendicular to the longitudinal direction. The lateral direction is shown in FIG. 22 as the “y” direction. The absorbent interlabial device 220 shown in FIG. 22 also has a transverse centerline T shown in FIG. 22. The “z” direction, shown in FIG. 22, is a direction parallel to the vertical plane described above. The term “upper” refers to an orientation in the z-direction toward the wearer's head. “Lower” or downwardly is toward the wearer's feet.

As shown in FIG. 21, the interlabial device 220 comprises at least a main absorbent portion 222 (or “central absorbent”). The main absorbent portion 222 includes an upper portion 226 and a lower portion 228 that is opposed to the upper portion. In use, the upper portion 226 is positioned furthest inward into the wearer's interlabial space. The main absorbent portion 222 also comprises a pair of ends 234. The main absorbent portion 222 should be at least partially absorbent. The main absorbent portion 222 may comprise non-absorbent portions, such as a liquid impervious barrier to prevent absorbed exudates from leaking out of the main absorbent portion 222.

The interlabial device 220 should be of a suitable size and shape that allows at least a portion thereof to fit comfortably within the wearer's interlabial space and to cover the wearer's vaginal orifice, and preferably also the wearer's urethra. The interlabial device 220 at least partially blocks, and more preferably completely blocks and intercepts the flow of menses, urine, and other bodily exudates from the wearer's vaginal orifice and urethra.

The main absorbent portion 222 of the embodiment shown in FIGS. 21-22 may comprise any suitable type of absorbent structure that is capable of absorbing and/or retaining liquids (e.g. menses and/or urine). The main absorbent portion 222 may be manufactured in a wide variety of shapes (as viewed from the side as in FIG. 21 or as viewed from the end as in FIG. 21A). Non limiting examples of shapes for the main absorbent portion when viewed from the end as in FIG. 21A. include ovoid, trapezoidal, rectangular, triangular, cylindrical, hemispherical or any combination of the above.

The main absorbent portion 222 may, likewise, be manufactured from a wide variety of liquid-absorbent materials commonly used in absorbent articles such as comminuted wood pulp which is generally referred to as airfelt. Examples of other suitable absorbent materials include cotton fibers or cotton lintels, creped cellulose wadding; meltblown polymers including coform; chemically stiffened, modified or cross-linked cellulosic fibers; synthetic fibers such as crimped polyester fibers; peat moss; tissue including tissue wraps and tissue laminates; absorbent foams; absorbent sponges; superabsorbent polymers (in fibrous and particulate form); absorbent gelling materials; or any equivalent material or combinations of materials, or mixtures of these. Preferred absorbent materials comprise folded tissues, cotton bafts, woven materials, nonwoven webs, needle punched rayon, and thin layers of foam. The main absorbent portion 222 may comprise a single material. Alternatively, as shown in FIG. 21A, the main absorbent portion 222 may comprise a combination of materials, such as a wrapping layer 246 surrounding a central wadding comprised of an absorbent material 244 (as shown in FIG. 22).

In the embodiment shown in FIG. 21, the main absorbent portion 222 is formed of a soft absorbent material such as rayon fibers or other suitable natural or synthetic fibers or sheeting. The main absorbent portion 222 shown in FIGS. 21-22 is generally of an ovoid cross sectional shape.

FIGS. 30-32 show another embodiment of an interlabial device comprising a transdermal administration system. The interlabial device is made of a hydrogel which has been cast into a spoon shape comprising a forward bowl-shaped part, which, upon use, is intended to be turned forward on the user and be brought over the user's clitoris, the bowl-shaped part facing the user's body. Moreover, the interlabial device comprises a rear-elongated part 303, which upon use is intended to be turned backwards on the user, and be arranged over the vagina.

The interlabial device 301 comprises a lower surface 304, which upon use is intended to be turned away from the user. Thus, the lower surface is substantially liquid-impermeable and may consist of a layer of a liquid-impermeable material such as a plastic film, or a coating of a liquid-impermeable material. Alternatively, the interlabial device can be manufactured in such a way that a substantially liquid-impermeable skin is formed on the lower surface.

Moreover, the interlabial device comprises an upper surface 305, which is intended to be facing the wearer upon use. The upper surface 305 receives and transports liquid into the interlabial device. Accordingly, the upper surface may be covered by a loose outer layer of a hydrophilic or a hydrophobic material. In case the liquid-receiving surface consists of a hydrogel surface, a slightly sticky, adhesive and humid surface is obtained, which is advantageous because the mucous membranes of the user will not be irritated. The interlabial device will also remain in place upon use. The upper surface 305 of the rear-elongated part is also covered with a transdermal administration system 380.

When the interlabial device is wetted upon use, the hydrogel is swelling and absorbing liquid, whereby a hump 306 is formed on the rear elongated part, as shown in FIGS. 31-32. Preferably, the transdermal administration system 380 is located on the hump 306. The shape and size of the hump is already determined when the interlabial device is manufactured because the interlabial device is cast in an anatomically designed mould. When the hydrogel swells, said gel also obtains a humid slightly sticky surface, which confers a soft and comfortable adhesion between the interlabial device and the wearer's body. It also keeps the mucous membranes of the interlabial space humid. The swollen hydrogel is soft and flexible as well as elastic, which means that the interlabial device is almost unnoticable to the wearer upon use.

FIGS. 33 and 34 show a sanitary napkin 401 according to a further embodiment of the invention.

Sanitary napkin 401 has a basically elongated shape with a longitudinal direction and a transverse direction. It has two long sides 402 and 403, two short sides 404 and 405, a first 406 and a second 407 end portion, a longitudinal centre line 408 extending over the product length and a transverse centre line 409 running laterally over the product width. The longitudinal centre line is considered to be a line extending in the longitudinal direction of the sanitary napkin equidistant from the long sides of the product. In the same way, the transverse centre line is a line arranged in the transverse direction of the napkin, equidistant between the short sides of the napkin. Napkin 401 has an upper side 410, intended to be turned towards the wearer during use, and a lower side 411 to be turned away from the wearer during use.

Sanitary napkin 1 incorporates a liquid-permeable outer layer 412 fixed on the side of the product to be turned towards the wearer (upper side 410) during use, and an impermeable rear layer 413 on the side of the napkin which will be turned away from the wearer (lower side 411) during use. Between the outer layer 412 and the impermeable rear layer 413 is an absorption body 414. The outer layer and the rear layer are connected at a joint outside the absorption body.

FIG. 34 shows a section through the napkin 1 in FIG. 33 along line II-II. On the lower side 411 of the napkin, on its impermeable layer 413, are placed the fasteners in the form of strips 415 made of pressure-sensitive adhesive arranged parallel with the longitudinal centre line 8 of the napkin. A removable protective layer 416 is laid on top of adhesive 415. Protective layer 416 is removed by the wearer before the towel is placed in the underwear. Other fastening methods can of course be used, such as Velcro® or friction-fastening.

Outer material 412 could be of any conventional material, for example non-woven, perforated plastic film or a laminate of these two materials. A transtermal administration system 480 is located on the outer material 412.

The most suitable material for the absorption body 414 is cellulose pulp. This can be made available as rolls, bales or sheets which, for the production of sanitary napkins, are dry-defibred and converted in fluffed form to a pulp matting, sometimes with an admixture of “superabsorbents” which are polymers with the power to absorb several times their own weight of water or body fluids. One alternative to this is to dry-form pulp matting as described in WO 94/10956. Examples of other usable absorption materials are various types of natural fibre such as cotton fibre, peat or similar. It is of course possible to use absorbent synthetic fibres, or mixtures of natural and synthetic fibres. Absorption body 414 can also include other materials such as form stabilizers, fluid-spreading means, or binders such as thermoplastic fibres which have been heat-treated to combine short fibres and particles into a continuous material. Various types of absorbent foam material can also be used in the absorption body.

The impermeable layer 413 (rear layer) consists of a liquid-impermeable material. Thin, fluid-tight plastic films are suitable for this purpose, but it is also possible to use material which is naturally permeable, but which is provided with a coating of plastic, resin or other fluid-tight material. In this way leakage of fluid from the lower side of the absorbent product is prevented. The impermeable layer 413 can therefore be made of any material which fulfills the impermeability criterion and offers sufficient flexibility and skin tolerance for this purpose. Examples of material suitable for use as an impermeable layer are plastic films, non-woven and laminates of these two. Plastic films can for instance be of polyethylene, polypropylene or polyester. The impermeable layer can alternatively consist of a laminate of an impermeable plastic layer, turned towards the absorption body, and a non-woven layer turned towards the underwear of the wearer. This type of construction provides a leakage-safe barrier layer with textile feel.

Between the outer layer 412 and the absorption body 414 is an acquisition layer 417. The purpose of this layer 417 is to draw fluid into the napkin and transmit it down to absorption body 414. This acquisition layer 417 can for example be a nonwoven material of low density.

It can be seen from FIG. 33 that the long sides 2 and 3 of the sanitary napkin are essentially arcuate. The arc shape is designed so that these long sides 2, 3 curve inwards towards the longitudinal centre line 8. The first end portion 6 has a width of 65 mm at its widest part. The widest part of the second end portion 7 is 30 mm.

1. An absorbent article, selected from the group consisting of a sanitary napkin, a panty liner, and an intralabial menstruation protection device, said article being suitable for administrating a pharmaceutical substance for treating, mitigating and/or preventing the premenstrual syndrome, said article comprising an absorbent body and having a generally elongated shape, having a first surface which is intended to face the wearer, and having a second surface which is intended to face away from the wearer characterised in that the first surface, which is intended to face the wearer, is at least partially covered with a transdermal therapeutic system containing a compound suitable for treating and/or preventing the premenstrual syndrome, whereby said compound is transdermally administrated to the wearer when said article is worn.

2. An absorbent article according to claim 1, characterised in that the article is an interlabial device.

3. An absorbent article according to claim 1, characterised in that the article is a sanitary napkin or a panty liner.

4. An absorbent article according to claim 3, characterised in that the artice has a hump on the surface intended to face the wearer.

5. An absorbent article according to claim 4, characterised in that the transdermal therapeutic system is arranged on the hump.

6. An absorbent article according to anyone of the proceeding claims, characterised in that the transdermal therapeutic system is a ceram, a lotion or an ointment.

7. An absorbent article according to anyone of claims 1-5, characterised in that the transdermal therapeutic system comprises one to three matrix layers that containing a pharmaceutical substance that can be used to treat and/or prevent premenstrual syndrome, together with matrix-forming compounds and optionally penetration-enhancing agents.

8. An absorbent article according to claim 7, characterised in that the transdermal therapeutic system also comprises a backing layer joined to the surface of the absorbent article that is intendede to face the wearer.

9. An absorbent article according to claim 8, characterised in that the transdermal therapeutic system is covered by a removable peeling layer.

10. An absorbent article according to anyone of the preceeding claims, characterised in that the pharmaceutical substance that can be used to treat and/or prevent premenstrual syndrome is chosen from the group of linoleic acid and metabolites, such as gamma-linolenic acid, dihomo-gamma linolenic acid, arachidonic acid, and adrenic acid, alpha-linolenic acid and metabolites, gestagens such as gestodene, levonogestrel, desogestrel, norethisterone, norethistosterone acetate and 3-keto-desogestrel, azaspirones or polycyclic amine derivatives including, among all, buspirone, gepirone, ipsapirone, and tandospirone, ergoline derivatives such as lisuride, bromolisuride, terguride, and proterguride.
